(19) United States
(12) Patent Application Publication
(10) Pub. No.: US 2005/0033391 A1
(43) Pub. Date: Feb. 10, 2005

(54) System and method for treating cardiac arrest and myocardial infarction

(75) Inventors: William J. Worthen, Coto de Caza, CA (US); Kenneth A. Collins, Mission Viejo, CA (US)

Correspondence Address: John L. Rogitz, Esq.;ROGITZ & ASSOCIATES, Suite 3120, 750 "B" Street, San Diego, CA, 92101, US

(73) Assignee: Alsius Corporation, Irvine, CA (US)

(21) Appl. No.: 10/913,080

(22) Filed: Aug. 6, 2004

Related U.S. Application Data
(60) Provisional application No. 60/492,818, filed on Aug. 6, 2003.

(51) Int. Cl. A61F 7/00; A61F 7/12

(52) U.S. Cl. 607/105; 607/113

(57) ABSTRACT

A closed loop heat exchange catheter can be placed in a patient suffering from cardiac arrest or myocardial infarction to cool the patient. Oxygen is infused into the patient to provide extra oxygen to the heart. Both modalities reduce infarct volume.

### RELATED APPLICATIONS

This application claims priority from U.S. provisional application Ser. No. 60/492,818 filed on Aug. 6, 2003.

### FIELD OF THE INVENTION

The present invention relates generally to systems for treating cardiac arrest and myocardial infarction.

### BACKGROUND OF THE INVENTION

Intravascular catheters have been introduced for controlling patient temperature. Typically, a coolant such as saline is circulated through an intravascular heat exchange catheter, which is positioned in the patient's bloodstream, to cool or heat the blood as appropriate for the patient's condition. The coolant is warmed or cooled by a computer-controlled heat exchanger that is external to the patient and that is in fluid communication with the catheter.

For example, intravascular heat exchange catheters can be used to combat potentially harmful fever in patients suffering from neurological and cardiac conditions such as stroke, subarachnoid hemorrhage, intracerebral hemorrhage, cardiac arrest, and acute myocardial infarction, or to induce therapeutic hypothermia in such patients. Further, such catheters can be used to rewarm patients after, e.g., cardiac surgery or for other reasons. Intravascular catheters afford advantages over external methods of cooling and warming, including more precise temperature control and more convenience on the part of medical personnel.

The following U.S. patents, all of which are incorporated herein by reference, disclose various intravascular catheters/systems/methods: 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559.

As critically recognized by the present invention, the provision of extra oxygen in combination with hypothermia can effectively treat cardiac arrest and AMI.

### SUMMARY OF THE INVENTION

A system for treating a patient includes a heat exchange catheter configured for placement in a patient to induce hypothermia in the patient when heat exchange fluid is circulated through the catheter. A heat exchanger supplies heat exchange fluid to the catheter and receives heat exchange fluid from the catheter in a closed circuit. A source of oxygen is used to deliver oxygen to the patient.

In one embodiment, the catheter has a heat exchange portion that is established by a balloon. In other embodiments, the heat exchange portion includes plural heat exchange fluid return tubes communicating with a supply lumen at a distal end of the catheter for carrying heat exchange fluid, with each return tube being formed spirally. In yet another embodiment, the heat exchange portion includes first and second elongated segments, each segment having an irregular exterior surface, and a flexible articulating joint connecting the first and second elongated segments.

In another aspect, a method for treating a patient includes inducing hypothermia in the patient using a closed loop heat exchange catheter, and simultaneously infusing oxygen to the patient.

In still another aspect, a system for treating a patient includes closed circuit heat exchange means and oxygen infusion means.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a perspective view of the present heat exchange catheter, schematically showing a medicament source and heat exchange fluid source in an exploded relationship with the catheter;

FIG. 2 is a perspective view of an alternate catheter; and

FIG. 3 is a perspective view of another alternate catheter, showing the distal portion of the catheter and an enlarged view of the heat exchange region.

### DETAILED DESCRIPTION OF THE PREFERRED EMBODIMENT

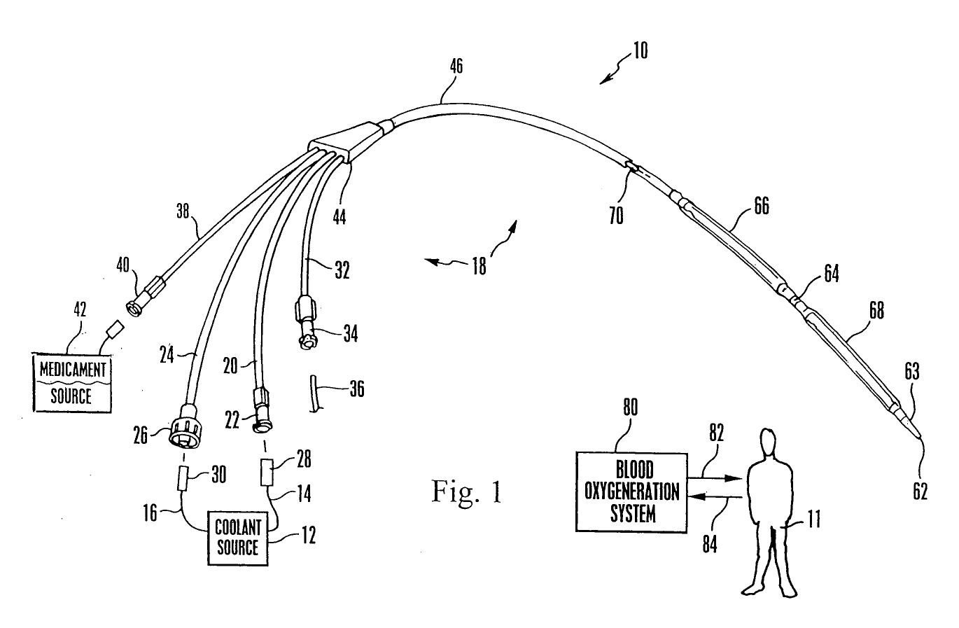

Referring initially to FIG. 1, a therapeutic catheter system, generally designated 10, is shown for establishing and maintaining hypothermia in a patient 11, or for attenuating a fever spike in a patient 11 and then maintaining normal body temperature in the patient. While FIG. 1 shows an exemplary embodiment of one heat exchange catheter, it is to be understood that the present invention applies to any of the catheters and accompanying heat exchangers disclosed in the above-referenced patents, including the helical shaped devices disclosed in Alsius' U.S. Pat. Nos. 6,451,045 and 6,520,933. Also, one of the spiral-shaped or convoluted-shaped catheters disclosed in Alsius' co-pending U.S. patent application Ser. Nos. 10/234,084, filed Aug. 30, 2002, for an “INTRAVASCULAR TEMPERATURE CONTROL CATHETER”, and in 10/355,776, filed Jan. 31, 2003, both of which are incorporated herein by reference, can be used.

Commencing the description of the system 10 at the proximal end, as shown the exemplary non-limiting system 10 includes a heat exchange fluid source 12 that can be a water-bath heat exchange system or a TEC-based heat exchange system such as any of the systems disclosed in one or more of the above-referenced patents. Or, the source 12 can be a source of compressed gas. In any case, the heat exchange fluid source provides warmed or cooled heat exchange fluid such as saline or compressed gas through a heat exchange fluid supply line 14, and heat exchange fluid is returned to the source 12 via a heat exchange fluid return line 16. A catheter, generally designated 18, includes a source tube 20 terminating in a fitting such as a female Luer fitting 22. Also, the catheter 18 has a return tube 24 terminating in a fitting such a male Luer fitting 26. The fittings 22, 26 can be selectively engaged with complementary fittings 28, 30 of the lines 14, 16 to establish a closed circuit heat exchange fluid path between the catheter 18 and heat exchange fluid source 12.

Additionally, a non-limiting catheter 18 may include a guide wire and primary infusion tube 32 that terminates in a fitting such as a female Luer 34. A guide wire 36 can be advanced through the tube 32 in accordance with central venous catheter placement principles, or medicament or other fluid can be infused through the guide wire and primary infusion tube 32. Moreover, a secondary infusion tube 38 with female Luer fitting 40 can be selectively engaged with a medicament source 42 for infusing fluid from the source 42 through the secondary tube 38 in accordance with present principles discussed below.

As discussed further below, the tubes 20, 24, 32, 38 are held in a distally-tapered connector manifold 44. As also set forth further below, the connector manifold 44 establishes respective pathways for fluid communication between the tubes 20, 24, 32, 38 and respective lumens in a catheter body 46.

In any case, the connector manifold 44 establishes a pathway for fluid communication between the heat exchange fluid supply tube 20 and the heat exchange fluid supply lumen of the catheter. Likewise, the connector manifold 44 establishes a pathway for fluid communication between the heat exchange fluid return tube 24 and the heat exchange fluid return lumen. Further, the connector manifold 44 establishes a pathway for fluid communication between the guide wire and primary infusion tube 32, and the guide wire lumen, which can terminate at an open distal hole 62 defined by a distally tapered and chamfered distal tip 63 of the catheter body 46. Also, the connector manifold 44 establishes a pathway for fluid communication between the secondary infusion tube 38 and the secondary infusion lumen, which can terminate at an infusion port 64 in a distal segment of the catheter body 46. Additional ports can be provided along the length of the catheter.

An exemplary non-limiting catheter 18 has a distally-located heat exchange member for effecting heat exchange with the patient 11 when the catheter is positioned in the vasculature or rectum or other orifice of a patient. The heat exchange member can be any of the heat exchange members disclosed in the above-referenced patents. By way of example, a non-limiting catheter shown in FIG. 1 can have proximal and distal thin-walled heat exchange membranes 66, 68 that are arranged along the last fifteen or so centimeters of the catheter body 46 and that are bonded to the outer surface of the catheter body 46, with the infusion port 64 being located between the heat exchange membranes 66, 68. Thus, each preferred non-limiting heat exchange membrane is about six centimeters to seven and one-half centimeters in length, with the heat exchange membranes being longitudinally spaced from each other along the catheter body 46 in the preferred embodiment shown. Essentially, the heat exchange membranes 66, 68 extend along most or all of that portion of the catheter 46 that is intubated within the patient. The heat exchange membranes can be established by a medical balloon material.

The heat exchange membranes 66, 68 can be inflated with heat exchange fluid from the heat exchange fluid source 12 as supplied from the heat exchange fluid supply lumen, and heat exchange fluid from the heat exchange membranes 66, 68 is returned via the heat exchange fluid return lumen to the heat exchange fluid source 12.

If desired, a temperature sensor 70 such as a thermistor or other suitable device can be attached to the catheter 18 as shown. The sensor 70 can be mounted on the catheter 18 by solvent bonding at a point that is proximal to the membranes 66, 68. Or, the sensor 70 can be disposed in a lumen of the catheter 18, or attached to a wire that is disposed in a lumen of the catheter 18, with the sensor hanging outside the catheter 18. Alternatively, a separate temperature probe can be used, such as the esophageal probe disclosed in U.S. Pat. No. 6,290,717, incorporated herein by reference. As yet another alternative, a rectal probe or tympanic temperature sensor can be used. In any case, the sensor is electrically connected to the heat exchange fluid source 12 for control of the temperature of the heat exchange fluid as described in various of the above-referenced patents.

As envisioned by the present invention, the structure set forth above can be used in many medical applications to cool a patient and/or to maintain temperature in a normothermic or hypothermic patient, for purposes of improving the medical outcomes of, e.g., cardiac arrest patients, patients suffering from myocardial infarction or stroke, etc. As another example, head trauma can be treated by and after lowering and maintaining the patient's temperature below normal body temperature. Preferably, particularly in the case of myocardial infarction, the heat exchange portions are advanced into the vena cava of the patient 11 to cool blood flowing to the heart.

Additionally, a blood oxygenation system 80 can be engaged with the patient 11 using, e.g., supply and return lines 82, 84 to provide oxygen to the patient 11. In one embodiment, the system 80 can be the system disclosed in U.S. Pat. No. 6,387,324, incorporated herein by reference, which extracorporeally adds oxygen to the blood of a patient and returns the oxygenated blood to the vasculature of the patient to supply supplementary oxygen to, e.g., the heart during or after an infarct episode to reduce the infarct volume. Or, the system 80 may be engaged with the patient by connecting the system 80 to the guide wire and primary infusion tube 32 and secondary infusion tube 38, such that blood is extracted from the patient through one of the tubes 32, 38, oxygenated by the system 80, and then returned to the patient through the other tube 38, 32. Yet again, the system 80 may be engaged with the patient's respiratory system through, e.g., an oxygen mask. Other ways known in the art to deliver oxygen may also be used.

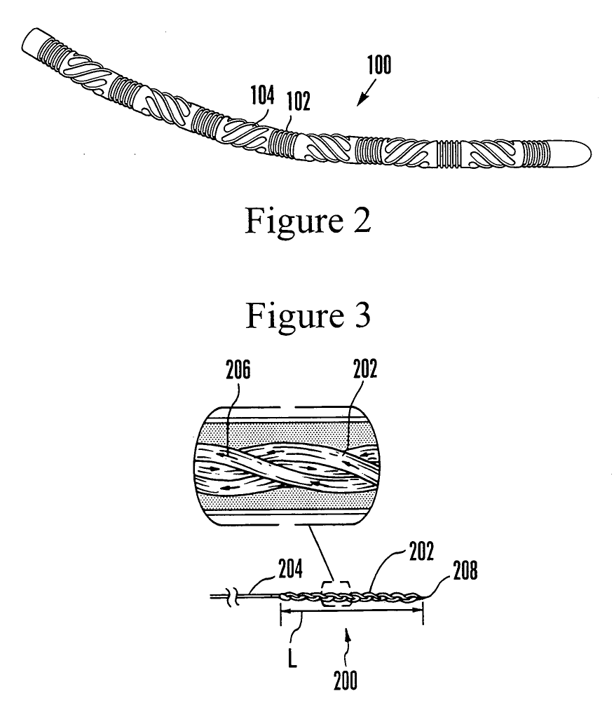

Now referring to FIG. 2, an alternate catheter 100 can include plural heat exchange elements 102. The heat exchange elements 102 can be established by one or more metal, preferably gold, hollow elongated segments that have external surfaces which have irregular exterior surfaces. Separating adjacent heat exchange elements 102 can be a flexible articulating joint 104, it being understood that the heat exchange elements 102 and joints 104 can be formed from a single piece of material such as plastic or metal, e.g., gold. The details of the heat exchange elements 102 and their configuration are set forth in U.S. Pat. No. 6,096,068, incorporated herein by reference. In any case, heat exchange fluid is circulated in a closed fluid communication loop between the heat exchange elements 102 and a heater/chiller to remove heat from the patient 12 to add heat to the patient to rewarm the patient after surgery or after the termination of therapeutic hypothermia treatment. When compressed gas is used as the heat exchange fluid, the gas is directed into the catheter, where it expands to cool the catheter and, thus, the patient.

FIG. 3 shows still another alternate heat exchange catheter 200. The catheter 200 shown in FIG. 3 can include plural heat exchange elements 202. The heat exchange elements 202 can be established by, e.g., three heat exchange fluid return tubes made of hollow plastic or metal, with each tube establishing a respective heat exchange fluid return lumen. A central heat exchange fluid supply lumen is established by a center tube 204. It is to be understood that the supply lumen conveys heat exchange fluid from a heater/chiller in a distal direction along the catheter 200, whereas the heat exchange elements 202 (the heat exchange fluid return tubes) convey heat exchange fluid back to the heater/chiller in a proximal direction as indicated by the arrows 206 in FIG. 3. Thus, heat exchange fluid is circulated in a closed fluid communication loop between the heat exchange elements 202 and heater/chiller to remove heat from the patient or to add heat to the patient to rewarm the patient after surgery or after the termination of therapeutic hypothermia treatment.

The heat exchange fluid return tubes are spirally formed around the center tube 204, and can be adhered thereto or not. That is, the preferred heat exchange elements 202 define spirals. The length “L” of the heat exchange region of the catheter 200 can be about 250 millimeters, with the pitch of the spiral heat exchange elements 202 being about 64 millimeters. In any case, the heat exchange fluid supply lumen terminates in a hollow distal tip 208, as do the lumens of the heat exchange elements 202. Accordingly, heat exchange fluid passes from the supply tube to the return tubes at the distal tip 208.

In operation, any one of the above-disclosed catheters is advanced (by, e.g., emergency response personnel) into the vasculature (preferably, the inferior vena cava or superior vena cava) or other cavity such as the rectum of a patient diagnosed as requiring temperature control. For example, a patient may be diagnosed with cardiac arrest, stroke, acute MI, or other malady for which therapeutic hypothermia may be indicated.

To cool the patient, the heat exchange fluid is cooled to below body temperature and circulated through the catheter as needed to reach a desired set point. Or, if the heat exchange fluid is gas, the gas is directed into the catheter where it expands and cools, cooling the catheter body. Oxygen is infused into the patient.

While the particular SYSTEM AND METHOD FOR TREATING CARDIAC ARREST AND MYOCARDIAL INFARCTION as herein shown and described in detail is fully capable of attaining the above-described objects of the invention, it is to be understood that it is the presently preferred embodiment of the present invention and is thus representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more”. All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. § 112, sixth paragraph, unless the element is expressly recited using the phrase “means for”.

1. A system for treating a patient, comprising:
a heat exchange catheter configured for placement in the patient to induce hypothermia in the patient when heat exchange fluid is circulated through the catheter;
a heat exchanger supplying heat exchange fluid to the catheter and receiving heat exchange fluid from the catheter in a closed circuit; and
a source of oxygen engageable with the patient to provide oxygen thereto for treatment of cardiac arrest or myocardial infarction.

2. The system of claim 1, wherein the heat exchange fluid is a compressed gas.

3. The system of claim 2, wherein a distal portion of the catheter includes at least one balloon.

4. The system of claim 2, wherein a distal portion of the catheter includes plural heat exchange fluid return tubes communicating with a supply lumen at a distal end of the catheter for carrying heat exchange fluid, each return tube being formed spirally.

5. The system of claim 2, wherein a distal portion of the catheter includes:
at least first and second elongated segments, each segment having an irregular exterior surface; and
a flexible articulating joint connecting the first and second elongated segments.

6. A method for reducing heart infarct volume in a patient, comprising:
inducing hypothermia in the patient using a closed loop heat exchange catheter placed in the patient; and
infusing oxygen into the patient to provide oxygen to the heart.

7. The method of claim 6, wherein the catheter includes at least one balloon.

8. The method of claim 6, wherein the catheter includes plural heat exchange fluid return tubes communicating with a supply lumen at a distal end of the catheter for carrying heat exchange fluid, each return tube being formed spirally.

9. The method of claim 6, wherein the catheter includes:
at least first and second elongated segments, each segment having an irregular exterior surface; and
a flexible articulating joint connecting the first and second elongated segments.

10. A system for treating a patient, comprising:
closed circuit heat exchange means configured for positioning in the patient to exchange heat therewith; and
oxygen infusion means for infusing extra oxygen to the patient's heart muscle.

11. The system of claim 10, wherein the heat exchange means includes at least one balloon.

12. The system of claim 10, wherein the heat exchange means includes plural heat exchange fluid return tubes communicating with a supply lumen at a distal end of the heat exchange means for carrying heat exchange fluid, each return tube being formed spirally.

13. The system of claim 10, wherein the heat exchange means includes:
at least first and second elongated segments, each segment having an irregular exterior surface; and
a flexible articulating joint connecting the first and second elongated segments.
